SEQUENCE LISTING (13 sequences)

SEQ ID NO: 1 - DNA, 1649 bp, Homo sapiens
gccacttcttctgggcccacgaggcagctgtcccatgctctgctgagcacggtggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttagctcactctgccatctgttttggcaagaccccacctccacgggctctcctgggccacccctgagtgcccagaccccaatccacagctctgggcttcctcggagacccctggggatggggatcttcagggaaggaactctggccacccaaacaggacaagagcagcctggggccaagcagacgggcaagtggagccacctctttcctccctccgcggatgaagcccagccacatttcagccgaggtccaaggcaggaggccatttacttgagacagattctctcctttttcctgtcccccatcttctctgggtccctctaacatctcccatggctctccccgcttctcctggtcactggagtctcctccccatgtacccaagg

SEQ ID NO: 2 - protein, 402 aa, Homo sapiens
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 3 - DNA, 6 bp, Artificial Sequence
  Description of Artificial Sequence Restriction site
tctaga

SEQ ID NO: 4 - DNA, 48 bp, Artificial Sequence
  Description of Artificial Sequence Primers
aactacccagtgggagcaccagaccacatctctgtgaagcagtgctag

SEQ ID NO: 5 - DNA, 52 bp, Artificial Sequence
  Description of Artificial Sequence Primers
aattctagcactgcttcacagagatgtggtctggtgctcccactgggtagtt

SEQ ID NO: 6 - DNA, 45 bp, Artificial Sequence
  Description of Artificial Sequence Primers
aactacccagtgggagcaccagaccacatctctgtgaagcagtag

SEQ ID NO: 7 - DNA, 49 bp, Artificial Sequence
  Description of Artificial Sequence Primers
aattctactgcttcacagagatgtggtctggtgctcccactgggtagtt

SEQ ID NO: 8 - DNA, 52 bp, Artificial Sequence
  Description of Artificial Sequence Primers
ctagacccgggatggcatccatgacaggaggacaacaaatggtaggccgtag

SEQ ID NO: 9 - DNA, 53 bp, Artificial Sequence
  Description of Artificial Sequence Primers
aattctacggcctacccatttgttgtcctcctgtcatggatgccatcccgggt

SEQ ID NO: 10 - DNA, 13 bp, Artificial Sequence
  Description of Artificial Sequence Primers
ctgcggccgcagt

SEQ ID NO: 11 - DNA, 17 bp, Artificial Sequence
  Description of Artificial Sequence Primers
ctagactgcggccgcag

SEQ ID NO: 12 - DNA, 1587 bp, Homo sapiens
  CDS (1)..(1584)
atgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcctgcggccgcagtctagagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatag

SEQ ID NO: 13 - protein, 528 aa, Homo sapiens
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVLRPQSRDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2004/0086519 A1
(43) Pub. Date: May 6, 2004

(54) Inhibition of stenosis or restenosis by P-selectin ligand antagonists

(75) Inventors: Anjali Kumar, Haverhill, MA (US); Robert G. Schaub, Pelham, NH (US); Jean-Francois Tanguay, Montreal (CA); Yahye Merhi, Dollard-des-Ormeaux (CA)

Correspondence Address: FINNEGAN, HENDERSON, FARABOW, GARRETT & DUNNER, LLP, 1300 I STREET, NW, WASHINGTON, DC, 20005, US

(73) Assignees: Genetics Institute, LLC; Montreal Heart Institute of Montreal

(21) Appl. No.: 10/431,359

(22) Filed: May 7, 2003

Related U.S. Application Data
(63) Continuation of application No. 09/660,047, filed on Sep. 12, 2000, now abandoned.

(51) Int. Cl. A61K 39/395; A61K 39/00

(52) U.S. Cl. 424/185.1; 424/145.1

(57) ABSTRACT

The present invention relates to methods and compositions for the modulation of restenosis and stenosis characterized by constrictive vascular remodeling and neointimal formation, in a subject, by administering a P-selectin antagonist. The invention further provides methods for modulating leukocyte recruitment, cell to cell adhesion, and cell adhesion to blood vessels in a subject by administering soluble P-selectin ligand, an anti-P-selectin ligand antibody, or an anti-P-selectin antibody. The invention also provides methods for identifying compounds capable of modulating restenosis.

### BACKGROUND OF THE INVENTION

Coronary artery disease is a major cause of morbidity and mortality in the Western world. The disease is typically manifested in intravascular stenosis (narrowing) or occlusion (blockage) due to atherosclerotic plaque. Percutaneous transluminal coronary balloon angioplasty (PTCA), for example, is widely used as the primary treatment for arteriosclerosis involving stenosis. PCTA is any percutaneous transluminal method of decreasing stenosis within a blood vessel. PTCA has an immediate success rate of more than 95%, but long term success remains limited by restenosis in 20-50% of patients within six months after intervention (Bult, H. (2000) Trends in Pharmacological Sciences 21:274-279). Stent implantation may improve the clinical outcome of PTCA, however, restenosis still remains a major clinical challenge. Indeed, restenosis is the single most significant problem in interventional cardiology and costs the health care system in excess of $1 billion per year.

Restenosis, the process of arterial re-narrowing, is a combination of neointimal formation and arterial remodeling in response to vascular injury such as that resulting from PTCA or other initially successful intervention. Vascular remodeling has a significant impact on chronic lumen area and may be responsible for 50% to 90% of late luminal area loss (Kumar, et al. (1997) Circulation 96(12):4333-4342). Remodeling is an adaptive process that occurs in response to chronic changes in hemodynamic conditions and may involve changes in many processes, such as cell growth, cell death, cell migration, and changes in extracellular matrix composition, that lead to a compensatory adjustment in vessel diameter and lumen area. The blood vessel is thought to remodel itself in response to long-term changes in flow, such that the lumen area is modified to maintain a predetermined level of shear stress (Kumar, et al. (1997) Circulation 96(12):4333-4342 and Orrego, et al (1999) Cardiologia 44(7)621).

Inflammatory reactions such as activation of granulocytes and neutrophils and platelet accumulation occur after PCTA. P-selectin rapidly appears on the cell surface of platelets when they are activated, mediating calcium-dependent adhesion of neutrophils or monocytes to platelets. P-selectin is also found in the Weibel-Palade bodies of endothelial cells; upon its release from these vesicles P-selectin mediates early binding of neutrophils to histamine-or thrombin-stimulated endothelium. In addition, selectins have been implicated in mediating interactions between endothelial cells and leukocytes in what is known as “leukocyte rolling,” which is generally believed to be the prerequisite for firm adhesion and subsequent transendothelial migration of leukocytes into tissues (Moore, (1998) Leuk Lymphoma 29(1-2): 1-15). Thus far three human selectin proteins have been identified, E-selectin (formerly ELAM-1), L-selectin (formerly LAM-1) and P-selectin (formerly PADGEM or GMP-140). The selectin proteins are characterized by a N-terminal lectin-like domain, an epidermal growth factor-like domain, and regions of homology to complement binding proteins.

Selectins are believed to mediate adhesion through specific interactions with ligands present on the surface of target cells. e.g. platelets. Generally the ligands of selectins are comprised at least in part of a carbohydrate moiety (e.g., sialyl Lewis^{x} (sLe^{x}) and sialyl Lewis^{a} (sLe^{a})). P-selectin binds to carbohydrates containing the non-sialated form of the Lewis^{x} blood group antigen and with higher affinity to sialyl Lewis^{x}. P-selectin Glycoprotein Ligand-1 (PSGL-1), a high-affinity P-selectin ligand, is expressed by leukocytes and platelets and mediates cell adhesion between these cell types (U.S. Pat. No. 5,843,707 and U.S. Pat. No. 5,827,817).

### SUMMARY OF THE INVENTION

The present invention provides methods and compositions for the modulation, (e.g., prevention, inhibition, and treatment) of stenosis and restenosis. The present invention is based, at least in part, on the discovery that P-selectin antagonism by P-selectin antagonists, including P-selectin ligand molecules, anti-P-selectin antibodies, and anti-P-selectin ligand antibodies inhibit cellular adhesion, e.g., platelet-leukocyte adhesion at the site of vascular injury, inhibit neointimal formation, and modulate vascular remodeling when administered to a subject with vascular injury or cardiodisease. The P-selectin moelcules of the invention are referred to herein as P-Selectin Glycoprotein Ligand-1 (PSGL-1) molecules.

Platelet-leukocyte adhesion, neointimal formation, and constrictive vascular remodeling all contribute to lumen loss and stenosis. Accordingly, P-selectin antagonists (e.g., PSGL-1 molecules, anti-P-selectin antibodies, and anti-P-selectin ligand antibodies) are useful agents in the modulation of stenosis and restenosis.

In one aspect, the invention provides a method for modulating stenosis and restenosis in a subject having vascular injury or cardiodisease, comprising administering a P-selectin antagonist. In one embodiment, the P-selectin antagonist is a P-selectin ligand protein. In a preferred embodiment, the P-selectin antagonist comprises a soluble P-selectin ligand protein, or a fragment thereof having P-selectin ligand activity, e.g., soluble PSGL-1 or a soluble recombinant PSGL fusion protein, e.g., rPSGL-Ig. In another embodiment, the P-selectin antagonist comprises an anti-P-selectin antibody or an anti-P-selectin ligand antibody. In a yet another embodiment, the composition further comprises a pharmaceutically acceptable carrier.

In one embodiment of the invention, stenosis and restenosis are characterized by constrictive vascular remodeling. In another embodiment, stenosis and restenosis are characterized by neointimal formation. In one embodiment the subject is a mammal, e.g., a human. In another embodiment, the P-selectin antagonist is administered to the subject prior to vascular injury. Vascular injury may result from, for example, coronary artery surgery, carotid artery surgery, angioplasty, e.g., percutaneous transluminal coronary angioplasty (PTCA), or implantation of one or more stents.

In another embodiment, the methods of the invention includes the administration of a soluble P-selectin ligand protein comprising at least a portion of an extracellular domain of a P-selectin ligand protein, for example, amino acid 42 to 60, 42 to 88, 42 to 118, 42 to 189, 42 to 310, or 42 to 316 of the amino acid sequence set forth in SEQ ID NO:2. In another embodiment, the protein comprises a soluble P-selectin ligand protein comprising at least an extracellular domain of a P-selectin ligand protein, for example, amino acids 21-316 of the amino acid sequence set forth in SEQ DI NO:2. In a further embodiment, the invention provides that the soluble protein comprises an Fc portion of an immunoglobulin, e.g., human IgG. In a related embodiment, the soluble protein comprises a soluble P-selectin ligand protein comprising the amino acid sequence from amino acid 42 to amino acid 60 of SEQ ID NO:2 fused at its C-terminus to the Fc portion of an immunoglobulin. In a related embodiment, the soluble protein comprises a soluble P-selectin ligand protein comprising the amino acid sequence from amino acid 42 to amino acid 88 of SEQ ID NO:2 fused at its C-terminus to the Fe portion of an immunoglobulin. In one embodiment, the amino acid sequence is fused through a linking sequence.

Another aspect of the invention provides a method for modulating leukocyte recruitment in a subject comprising administering a P-selectin antagonist, e.g., a PSGL-1, anti-P-selectin ligand antibody, or an anti-P-selectin antibody. Yet another aspect of the invention provides a method for inhibiting cell to cell adhesion in a subject comprising administering a P-selectin antagonist, e.g., soluble PSGL-1, an anti-P-selectin ligand antibody, or an anti-P-selectin antibody. In one embodiment, the adhesive cells are selected from the group consisting of leukocytes, platelets, and endothelial cells. A further aspect of the invention provides a method for inhibiting cell adhesion to blood vessels in a subject comprising administering a P-selectin antagonist, e.g., soluble PSGL-1, an anti-P-selectin ligand antibody, or an anti-P-selectin antibody, thereby inhibiting cell adhesion to blood vessels. In one embodiment, the adhesive cells are selected from the group consisting of leukocytes, platelets and endothelial cells.

In yet another aspect, the invention provides a method for identifying a compound capable of modulating stenosis or restenosis comprising assaying the ability of the compound to modulate PSGL-1 protein activity. In one embodiment, the ability of the compound to modulate PSGL-1 polypeptide activity is determined by detecting a decrease in intercellular adhesion. For example, cellular adhesion may involve leukocytes, endothelial cells, or platelets. In another embodiment, the ability of the compound to modulate PSGL-1 polypeptide activity is determined by detecting positive vascular remodeling after vascular injury. In yet another embodiment, the ability of the compound to modulate PSGL-1 polypeptide activity is determined by detecting a reduction of neointimal formation.

Other features and advantages of the invention will be apparent from the following detailed description and claims.

### BRIEF DESCRIPTION OF THE DRAWINGS

FIG. 1 is a graph depicting platelet adhesion to deeply damaged arterial segments at 1 and 4 hours and at 1 and 4 weeks post-angioplasty in control and rPSGL-Ig-treated pigs.

FIG. 2 is a graph depicting neutrophil adhesion to deeply damaged arterial segments at 1 and 4 hours and 1 and 4 weeks post-angioplasty in control and rPSGL-Ig-treated pigs.

FIG. 3 is a graph illustrating the correlation between vascular stenosis and normalized external elastic lamina (EEL) surface in control and rPSGL-Ig-treated pig arteries at 4 weeks.

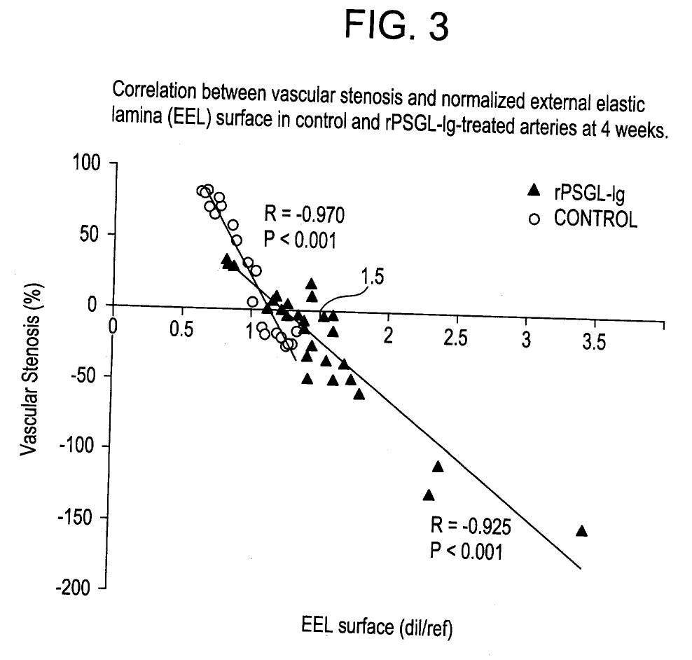

FIG. 4 is a graph illustrating the correlation between vascular stenosis and neointimal surface area in control and rPSGL-Ig-treated pig arteries at 4 weeks.

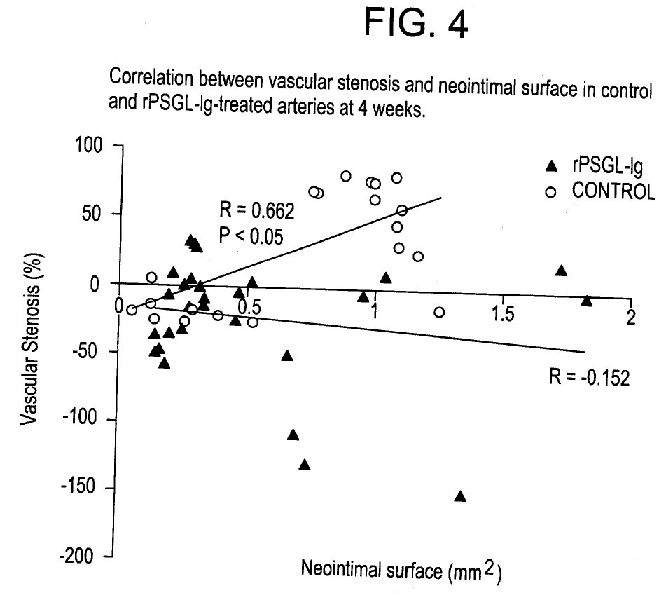

FIG. 5 is a graph depicting the external elastic lumina (EEL) surface and residual lumen in control and rPSGL-Ig-treated pig arteries at 1 and 4 weeks.

FIG. 6 depicts the morphology of arterial sections and expression of P-selectin by neoendothelium at 4 weeks in control and rPSGL-Ig-treated pig arteries.

### DETAILED DESCRIPTION OF THE INVENTION

The present invention provides methods and compositions for the modulation, e.g., treatment, inhibition, or prevention of stenosis or restenosis, in vivo, by administration of P-selectin antagonists, e.g., P-selectin ligand molecules, anti-P-selectin antibodies, and anti-P-selectin ligand antibodies. The P-selectin ligand molecules used in the methods of the invention are referred to herein as P-Selectin Glycoprotein Ligand-1 (PSGL-1) molecules.

The P-selectin antagonists of the methods of the invention can be used to modulate cell-cell adhesion, e.g., platelet-leukocyte adhesion, neointimal formation, and vascular remodeling in a subject where the subject has vascular injury resulting from cardiovascular disease, e.g., arteriosclerosis, or non-pathologic vascular intervention, e.g., PCTA or stent implantation, and are, accordingly, useful in modulating stenosis or restenosis. In another embodiment, the P-selectin antagonists of the methods of the invention can be used to modulate restenosis in a subject where the subject has vascular injury and subsequently undergoes vascular intervention, such as angioplasty or stent implantation.

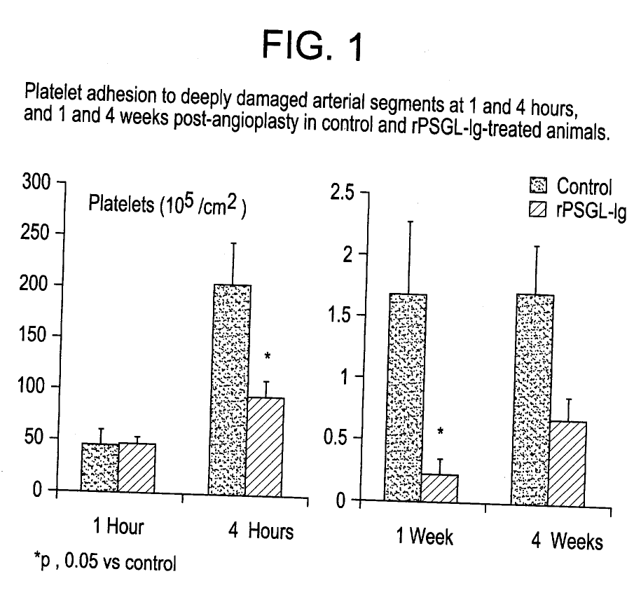

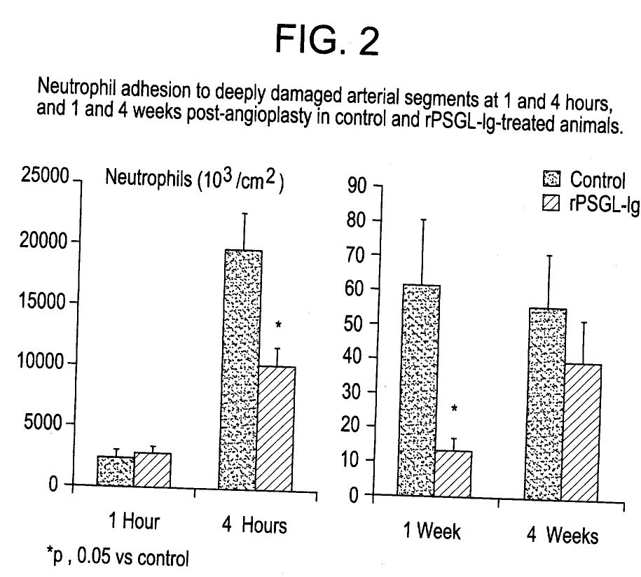

Platelets and leukocytes adhere to damaged arterial walls after vascular injury through binding between P-selectin and PSGL-1, which is expressed on leukocytes and platelets. In one aspect of the invention, antagonism of P-selectin by a P-selectin ligand, an anti-P-selectin antibody, or an anti-P-selectin ligand antibody inhibits both platelet and neutrophil adhesion to damaged arterial segments, thereby modulating restenosis (see Example 2 and FIGS. 1 and 2). Interaction between platelets and leukocytes, e.g., intercellular adhesion, is also inhibited by the P-selectin antagonists of the invention.

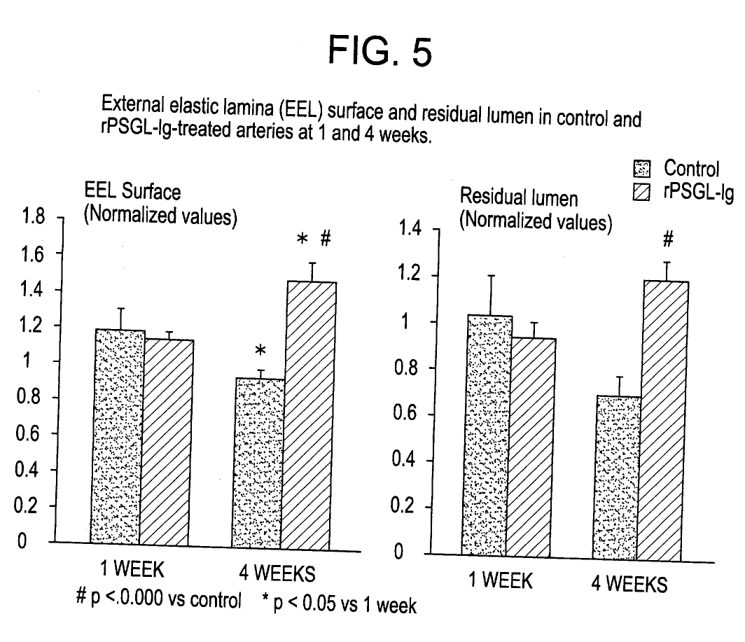

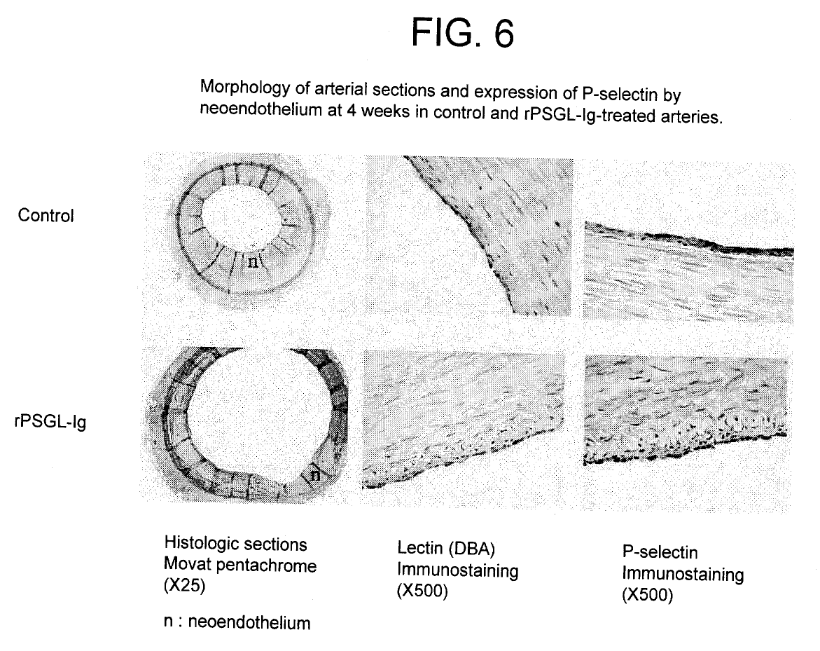

In another aspect of the invention, administering a P-selectin antagonist to a subject with vascular injury, where the subject is undergoing subsequent vascular intervention resulting in further vascular injury, results in a larger residual lumen and a larger external elastic lumina (EEL) post-injury (see Table 2 in Example 2 and FIGS. 5 and 6), compared to a control, thereby positively impacting vascular remodeling. As demonstrated herein, in animals having prior vascular injury that are treated with a P-selectin antagonist, e.g., soluble rPSGL-Ig, prior to a second vascular injury, the percentage of vascular restenosis is significantly less (p<0.005) than in control animals. Accordingly, restenosis is modulated, e.g., prevented, inhibited, or treated, by administration of a P-selectin antagonist (see Examples 2). In yet another aspect of the invention, P-selectin antagonism inhibits neointimal formation. In one embodiment, a subject who has prior vascular injury and is undergoing stent implantation is treated by the administration of a P-selectin antagonist to thereby inhibit neotintimal formation in the subject. Accordingly, stenosis and restenosis are inhibited (see Example 3).

As used herein, a “P-selectin antagonist” includes any agents which are capable of antagonizing P-selectin, e.g., by inhibiting interaction between P-selectin and a P-selectin ligand protein, e.g., by inhibiting interaction of P-selectin expressing platelets with PSGL expressing leukocytes. For example, P-selectin antagonists include anti-P-selectin antibodies, anti-P-selectin ligand antibodies, P-selectin ligand molecules, e.g. PSGL-1, or fragments thereof having P-selectin ligand activity as well as small molecules. In a preferred embodiment, the P-selectin ligand is soluble.

As used interchangeably herein, “P-selectin ligand activity,” “PSGL-1 activity,” “biological activity of PSGL-1” or “functional activity of PSGL-1” includes an activity exerted by a PSGL-1 protein, polypeptide or nucleic acid molecule on a PSGL-1 responsive cell, e.g., platelet, leukocyte, or endothelial cell, as determined in vivo, or in vitro, according to standard techniques. PSGL-1 activity can be a direct activity, such as an association with a PSGL-1-target molecule e.g., P-selectin. As used herein, a “substrate” or “target molecule” or “binding partner” is a molecule, e.g. P-selectin, with which a PSGL-1 protein binds or interacts in nature, such that PSGL-1-mediated function, e.g., modulation of cell migration or adhesion, is achieved. A PSGL-1 target molecule can be a non-PSGL-1 molecule or a PSGL-1 protein or polypeptide. Examples of such target molecules include proteins in the same signaling path as the PSGL-1 protein, e.g., proteins which may function upstream (including both stimulators and inhibitors of activity) or downstream of the PSGL-1 protein in a pathway involving regulation of P-selectin binding. Alternatively, a PSGL-1 activity is an indirect activity, such as a cellular signaling activity mediated by interaction of the PSGL-1 protein with a PSGL-1 target molecule. The biological activities of PSGL-1 are described herein, and include, for example, one or more of the following activities: 1) binding to or interacting with P-selectin; 2) modulating P-selectin binding; 2) modulating intercellular adhesion, e.g., platelet-leukocyte adhesion and endothelial-leukocyte adhesion; 3) modulating cell migration, e.g., leukocyte recruitment to platelets and endothelial cells; 4) modulating stenosis or restenosis; 5) modulating vascular remodeling; and 6) modulating neointimal formation.

As used herein, “stenosis” includes the process of arterial narrowing. “Restenois” includes the process of arterial re-narrowing following initially successful vascular intervention. Stenosis and restenosis are characterized by neointimal formation (e.g., intimal thickening), and constrictive vascular remodeling in response to vascular injury such as that resulting from percutaneous transluminal coronary angioplasty (PTCA) or other initially successful intervention (restenosis), or in response to vascular injury resulting from pathogenic stimuli, e.g., vascular or cardiovascular disease (stenosis). Vascular remodeling is an adaptive process of structural changes in vascular wall structures, and involves changes in many processes, such as cell growth, cell death, cell migration, intercellular adhesion, and changes in extracellular matrix composition, that lead to a compensatory adjustment in vessel diameter and lumen area. In the context of restenosis or stenosis, vascular remodeling refers to a loss of lumen area by a combination of reduction in vessel diameter and neointimal thickening (e.g., constrictive vascular remodeling). As used herein, “positive vascular remodeling” includes an increase in the lumen area of a vessel, or an increase in vessel diameter. Positive vascular remodeling also includes a lack of constrictive vascular remodeling, e.g., a decrease in lumen area of a vessel or a decrease in vessel diameter, after vascular injury caused by vascular intervention or vascular disease.

A subject who may be at risk for stenosis is one who suffers from a cardiovascular disease or disorder. A subject who may be at risk for restenosis is one who is undergoing cardiovascular or general vascular procedures or intervention such as angioplasty of any vessel, e.g., carotid, femoral, coronary, etc.; surgical revascularization, e.g., balloon angioplasty, laser angioplasty, percutaneous transluminal coronary angioplasty (PTCA), coronary artery bypass grafting, rotational atherectomy or coronary artery stents, or other intervention, surgical or non-surgical, which may cause vascular injury. Administration of a P-selectin antagonist to modulate restenosis may be prior to injury, during the intervention procedure, or after the injury or intervention has occurred. In a preferred embodiment, administration of the P-selectin antagonist is prior to surgical intervention.

The P-selectin ligand molecules used in the methods of the invention are described in U.S. Pat. Nos. 5,827,817, 5,840,679, and 5,843,707, the contents of which are incorporated herein by reference. PGSL-1 is a glycoprotein which acts as a ligand for P-selectin on endothelial cells and platelets. The DNA sequence of PSGL-1 is set forth in SEQ ID NO:1. The complete amino acid sequence of the PSGL-1, i.e., the mature peptide plus the leader sequence, is characterized by the amino acid sequence set forth in SEQ ID NO:2 from amino acid 1 to amino acid 402. The mature PSGL-1 protein is characterized by the amino acid sequence set forth in SEQ ID NO:2 from amino acid 42 to amino acid 402.

As used herein, a “soluble PSGL-1 protein,” or a “soluble P-selectin ligand protein,” refers to a soluble P-selectin ligand glycoprotein, e.g., soluble PSGL-1, or a fragment thereof having a P-selectin ligand activity, which includes a carbohydrate comprising sLe^{x}. Soluble P-selectin ligand proteins used in the methods of the invention preferably include at least an extracellular domain of PSGL-1, e.g., about amino acid 21 to about amino acid 316 of SEQ ID NO:2. Other soluble forms of the P-selectin ligand molecules are characterized by the amino acid sequence set forth in SEQ ID NO:2 from amino acids 42 to 310. In one embodiment of the methods of the invention, soluble forms of the P-selectin ligand molecules of the methods of the invention may be fused through “linker” sequences to the Fc portion of an immunoglobulin, e.g., an IgG molecule (see Example 1D). In another embodiment of the invention, the soluble P-selectin ligand protein is a chimeric molecule which is comprised of the extracellular domain of a PSGL-1 protein molecule, a carbohydrate comprising sLe^{x}, and is fused through linker sequences to the Fc portion of human IgG. This soluble form of PSGL-1 is referred to herein as soluble rPSGL-Ig.

The methods of the invention encompass the use of nucleic acid molecules that differ from the nucleotide sequence shown in SEQ ID NO:1 due to degeneracy of the genetic code and thus encode the same PSGL-1 proteins as those encoded by the nucleotide sequence shown in SEQ ID NO:1. In another embodiment, an isolated nucleic acid molecule included in the methods of the invention has a nucleotide sequence encoding a protein having an amino acid sequence shown in SEQ ID NO:2.

The methods of the invention further include the use of allelic variants of human PSGL-1, e.g., functional and non-functional allelic variants. Functional allelic variants are naturally occurring amino acid sequence variants of the human PSGL-1 protein that maintain a PSGL-1 activity as described herein, e.g., P-selectin binding. Functional allelic variants will typically contain only conservative substitution of one or more amino acids of SEQ ID NO:2, or substitution, deletion or insertion of non-critical residues in non-critical regions of the protein. Non-functional allelic variants are naturally occurring amino acid sequence variants of the human PSGL-1 protein that do not have a PSGL-1 activity. Non-functional allelic variants will typically contain a non-conservative substitution, deletion, or insertion or premature truncation of the amino acid sequence of SEQ ID NO:2, or a substitution, insertion or deletion in critical residues or critical regions of the protein.

Various aspects of the invention are described in further detail in the following subsections:

I. Isolated PSGL-1 Proteins, Anti-PSGL-1 Antibodies, and Anti-P-Selectin Antibodies Used in the Methods of the Invention

The methods of the invention include the use of isolated P-selectin ligand proteins, e.g. PGSL-1 proteins, and biologically active portions thereof, as well as polypeptide fragments suitable for use as immunogens to raise anti-P-selectin ligand antibodies. In one embodiment, native PSGL-1 proteins can be isolated from cells or tissue sources by an appropriate purification scheme using standard protein purification techniques. In another embodiment, PSGL-1 proteins are produced by recombinant DNA techniques. Alternative to recombinant expression, a PSGL-1 protein or polypeptide can be synthesized chemically using standard peptide synthesis techniques.

As used herein, a “biologically active portion” of a PSGL-1 protein includes a fragment of a PSGL-1 protein having a PSGL-1 activity. Biologically active portions of a PSGL-1 protein include peptides comprising amino acid sequences sufficiently identical to or derived from the amino acid sequence of the PSGL-1 protein, e.g., the amino acid sequence shown in SEQ ID NO:2, which include fewer amino acids than the full length PSGL-1 proteins, and exhibit at least one activity of a PSGL-1 protein. Typically, biologically active portions comprise a domain or motif with at least one activity of the PSGL-1 protein (e.g., a fragment containing amino acids 42 to 60 of SEQ ID NO:2 is capable of interacting with P-selectin). A biologically active portion of a PSGL-1 protein can be a polypeptide which is, for example, 25, 50, 75, 100, 125, 150, 175, 200, 250, 300 or more amino acids in length. Biologically active portions of a PSGL-1 protein can be used as targets for developing agents which modulate a PSGL-1 activity.

In a preferred embodiment, the PSGL-1 protein used in the methods of the invention has at least an extracellular domain of the amino acid sequence shown in SEQ ID NO:2 or P-selectin binding fragment of the extracellular domain of PSGL-1, or an extracellular domain of SEQ ID NO:2. In other embodiments, the PSGL-1 protein is substantially identical to SEQ ID NO:2, and retains the functional activity of the protein of SEQ ID NO:2, yet differs in amino acid sequence due to natural allelic variation or mutagenesis, as described in detail in subsection II below. Accordingly, in another embodiment, the PSGL-1 protein used in the methods of the invention is a protein which comprises an amino acid sequence at least about 50%, 55%, 60%, 65%, 70%, 75%, 80%, 85%, 90%, 95%, 96% 97%, 98%, 99% or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the PSGL-1 amino acid sequence of SEQ ID NO:2 having 1600 amino acid residues, at least 480, preferably at least 640, more preferably at least 800, even more preferably at least 960, and even more preferably at least 1120, 1280, or 1440 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use PSGL-1 chimeric or fusion proteins. As used herein, a PSGL-1 “chimeric protein” or “fusion protein” comprises a PSGL-1 polypeptide operatively linked to a non-PSGL-1 polypeptide. A “PSGL-1 polypeptide” refers to a polypeptide having an amino acid sequence corresponding to a PSGL-1 molecule, whereas a “non-PSGL-1 polypeptide” refers to a polypeptide having an amino acid sequence corresponding to a protein which is not substantially homologous to the PSGL-1 protein, e.g., a protein which is different from the PSGL-1 protein and which is derived-from the same or a different organism. Within a PSGL-1 fusion protein the PSGL-1 polypeptide can correspond to all or a portion of a PSGL-1 protein. In a preferred embodiment, a PSGL-1 fusion protein comprises at least one biologically active portion of a PSGL-1 protein, e.g., an extracellular domain of PSGL-1 or P-selectin binding fragment thereof. In another preferred embodiment, a PSGL-1 fusion protein comprises at least two biologically active portions of a PSGL-1 protein. Within the fusion protein, the term “operatively linked” is intended to indicate that the PSGL-1 polypeptide and the non-PSGL-1 polypeptide are fused in-frame to each other. The non-PSGL-1 polypeptide can be fused to the N-terminus or C-terminus of the PSGL-1 polypeptide.

For example, in one embodiment, the fusion protein is a recombinant soluble form of PSGL-1 protein in which the extracellular domain of the PSGL-1 molecule is fused to human IgG, e.g., soluble rPSGL-Ig.

In another embodiment, this fusion protein is a PSGL-1 protein containing a heterologous signal sequence at its N-terminus. In certain host cells (e.g., mammalian host cells), expression and/or secretion of PSGL-1 can be increased through use of a heterologous signal sequence.

The soluble PSGL-1 fusion proteins used in the methods of the invention, e.g. rPSGL-Ig, can be incorporated into pharmaceutical compositions and administered to a subject in vivo. The soluble PSGL-1 fusion proteins can be used to affect the bioavailability of a PSGL-1 substrate, e.g., P-selectin.

Moreover, the PSGL-1-fusion proteins used in the methods of the invention can be used as immunogens to produce anti-P-selectin ligand antibodies in a subject, to purify P-selectin ligands and in screening assays to identify molecules which inhibit the interaction of a P-selectin ligand molecule with a P-selectin molecule.

Preferably, a PSGL-1 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A PSGL-1-encoding nucleic acid can be cloned into such an expression vector such that the fusion moiety is linked in-frame to the PSGL-1 protein.

The present invention also pertains to the use of variants of the PSGL-1 proteins which function as either PSGL-1 agonists (mimetics) or as PSGL-1 antagonists. Variants of the PSGL-1 proteins can be generated by mutagenesis, e.g., discrete point mutation or truncation of a PSGL-1 protein. An agonist of the PSGL-1 proteins can retain substantially the same, or a subset, of the biological activities of the naturally occurring form of a PSGL-1 protein. An antagonist of a PSGL-1 protein can inhibit one or more of the activities of the naturally occurring form of the PSGL-1 protein by, for example, competitively modulating a PSGL-1-mediated activity of a PSGL-1 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the PSGL-1 protein.

In one embodiment, variants of a PSGL-1 protein which function as either PSGL-1 agonists (mimetics) or as PSGL-1 antagonists can be identified by screening combinatorial libraries of mutants, e.g., truncation mutants, of a PSGL-1 protein for PSGL-1 protein agonist or antagonist activity. In one embodiment, a variegated library of PSGL-1 variants is generated by combinatorial mutagenesis at the nucleic acid level and is encoded by a variegated gene library. A variegated library of PSGL-1 variants can be produced by, for example, enzymatically ligating a mixture of synthetic oligonucleotides into gene sequences such that a degenerate set of potential PSGL-1 sequences is expressible as individual polypeptides, or alternatively, as a set of larger fusion proteins (e.g., for phage display) containing the set of PSGL-1 sequences therein. There are a variety of methods which can be used to produce libraries of potential PSGL-1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PSGL-1 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a PSGL-1 protein coding sequence can be used to generate a variegated population of PSGL-1 fragments for screening and subsequent selection of variants of a PSGL-1 protein. In one embodiment, a library of coding sequence fragments can be generated by treating a double stranded PCR fragment of a PSGL-1 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the PSGL-1 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of PSGL-1 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify PSGL-1 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-PSGL-1 antibodies and anti-P-selectin antibodies. An isolated PSGL-1 protein, or P-selectin protein, or a portion or fragment thereof, can be used as an immunogen to generate antibodies that bind PSGL-1 or P-selectin using standard techniques for polyclonal and monoclonal antibody preparation. A full-length PSGL-1 protein or P-selectin protein can be used or, alternatively, antigenic peptide fragments of PSGL-1 or P-selectin can be used as immunogens (Johnston et al. Cell 56: 1033-1044 1989). The antigenic peptide of PSGL-1 comprises at least 8 amino acid residues of the amino acid sequence shown in SEQ ID NO:2 and encompasses an epitope of PSGL-1 such that an antibody raised against the peptide forms a specific immune complex with the PSGL-1 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of PSGL-1 that are located on the surface of the protein, e.g., hydrophilic regions, as well as regions with high antigenicity.

A PSGL-1 or P-selectin immunogen is typically used to prepare antibodies by immunizing a suitable subject, (e.g., rabbit, goat, mouse, or other mammal) with the immunogen. An appropriate immunogenic preparation can contain, for example, recombinantly expressed PSGL-1 protein or P-selectin protein or a chemically synthesized PSGL-1 or P-selectin polypeptide. The preparation can further include an adjuvant, such as Freund's complete or incomplete adjuvant, or similar immunostimulatory agent. Immunization of a suitable subject with an immunogenic PSGL-1 preparation induces a polyclonal anti-PSGL-1 or anti-P-selectin antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a PSGL-1 or P-selectin. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind PSGL-1 molecules. The term “monoclonal antibody” or “monoclonal antibody composition”, as used herein, refers to a population of antibody molecules that contain only one species of an antigen binding site capable of immunoreacting with a particular epitope of PSGL-1. A monoclonal antibody composition thus typically displays a single binding affinity for a particular PSGL-1 protein with which it immunoreacts.

Polyclonal anti-PSGL-1 antibodies can be prepared as described above by immunizing a suitable subject with a PSGL-1 immunogen. The anti-PSGL-1 antibody titer in the immunized subject can be monitored over time by standard techniques, such as with an enzyme linked immunosorbent assay (ELISA) using immobilized PSGL-1. If desired, the antibody molecules directed against PSGL-1 can be isolated from the mammal (e.g., from the blood) and further purified by well known techniques, such as protein A chromatography to obtain the IgG fraction. At an appropriate time after immunization, e.g., when the anti-PSGL-1 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a PSGL-1 immunogen as described above, and the culture supernatants of the resulting hybridoma cells are screened to identify a hybridoma producing a monoclonal antibody that binds PSGL-1.

Any of the many well known protocols used for fusing lymphocytes and immortalized cell lines can be applied for the purpose of generating an anti-PSGL-1 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind PSGL-1, e.g., using a standard ELISA assay.

Alternative to preparing monoclonal antibody-secreting hybridomas, a monoclonal anti-PSGL-1 antibody can be identified and isolated by screening a recombinant combinatorial immunoglobulin library (e.g., an antibody phage display library) with PSGL-1 to thereby isolate immunoglobulin library members that bind PSGL-1. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-PSGL-1 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the alt, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-PSGL-1 antibody can be used to detect PSGL-1 protein (e.g., in a cellular lysate or cell supernatant) in order to evaluate the abundance and pattern of expression of the PSGL-1 protein. Anti-PSGL-1 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

II. Isolated Nucleic Acid Molecules Used in the Methods of the Invention

The coding sequence of the isolated human PSGL-1 cDNA and the amino acid sequence of the human PSGL-1 polypeptide are shown in SEQ ID NOs:1 and 2, respectively. The PSGL-1 sequence is also described in U.S. Pat. Nos. 5,827,817, 5,840,679, and 5,843,707, the contents of which are incorporated herein by reference.

The methods of the invention include the use of isolated nucleic acid molecules that encode PSGL-1 proteins or biologically active portions thereof, as well as nucleic acid fragments sufficient for use as hybridization probes to identify PSGL-1-encoding nucleic acid molecules (e.g., PSGL-1 mRNA) and fragments for use as PCR primers for the amplification or mutation of PSGL-1 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, or a portion thereof, can be isolated using standard molecular biology techniques and the sequence information provided herein. Using all or portion of the nucleic acid sequence of SEQ ID NO:1 as a hybridization probe, PSGL-1 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to PSGL-1 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, a complement of the nucleotide sequence shown in SEQ ID NO:1, or a portion of any of these nucleotide sequences. A nucleic acid molecule which is complementary to the nucleotide sequence shown in SEQ ID NO:1, is one which is sufficiently complementary to the nucleotide sequence shown in SEQ ID NO:1 such that it can hybridize to the nucleotide sequence shown in SEQ ID NO:1 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:1 or a portion of any of this nucleotide sequence.

Moreover, the nucleic acid molecules used in the methods of the invention can comprise only a portion of the nucleic acid sequence of SEQ ID NO:1, for example, a fragment which can be used as a probe or primer or a fragment encoding a portion of a PSGL-1 protein, e.g., a biologically active portion of a PSGL-1 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 of an anti-sense sequence of SEQ ID NO:1 or of a naturally occurring allelic variant or mutant of SEQ ID NO:1. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, or more nucleotides in length and hybridizes under stringent hybridization conditions to a nucleic acid molecule of SEQ ID NO:1.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4×sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a PSGL-1 protein, such as by measuring a level of a PSGL-1-encoding nucleic acid in a sample of cells from a subject e.g., detecting PSGL-1 mRNA levels or determining whether a genomic PSGL-1 gene has been mutated or deleted.

The methods of the present invention may use non-human orthologues of the human PSGL-1 protein. Orthologues of the human PSGL-1 protein are proteins that are isolated from non-human organisms and possess the same PSGL-1 activity.

The methods of the present invention further include the use of nucleic acid molecules comprising the nucleotide sequence of SEQ ID NO:1 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of PSGL-1 (e.g., the sequence of SEQ ID NO:2) without altering the biological activity, whereas an “essential” amino acid residue is required for biological activity. For example, amino acid residues comprising fragments which are capable of interacting with P-selectin or which are capable of inhibiting P-selectin-mediated intercellular adhesion or cellular migration are not likely to be amenable to alteration.

Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a PSGL-1 protein is preferably replaced with another amino acid residue from the same side chain family. Alternatively, in another embodiment, mutations can be introduced randomly along all or part of a PSGL-1 coding sequence, such as by saturation mutagenesis, and the resultant mutants can be screened for PSGL-1 biological activity to identify mutants that retain activity. Following mutagenesis of SEQ ID NO:1 the encoded protein can be expressed recombinantly and the activity of the protein can be determined using the assay described herein.

Given the coding strand sequences encoding PSGL-1 disclosed herein, antisense nucleic acids of the invention can be designed according to the rules of Watson and Crick base pairing. The antisense nucleic acid molecule can be complementary to the entire coding region of PSGL-1 mRNA, but more preferably is an oligonucleotide which is antisense to only a portion of the coding or noncoding region of PSGL-1 mRNA. For example, the antisense oligonucleotide can be complementary to the region surrounding the translation start site of PSGL-1 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the PSGL-1 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of PSGL-1 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of PSGL-1 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of PSGL-1 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of PSGL-1 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5:1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

III Recombinant Expression Vectors and Host Cells Used in the Methods of the Invention

The methods of the invention (e.g., the screening assays described herein) include the use of vectors, preferably expression vectors, containing a nucleic acid encoding a PSGL-1 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., PSGL-1 proteins, mutant forms of PSGL-1 proteins, fusion proteins, and the like).

The recombinant expression vectors to be used in the methods of the invention can be designed for expression of P-selectin ligand proteins in prokaryotic or eukaryotic cells. For example, PSGL-1 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in PSGL-1 activity assays, (e.g., direct assays or competitive assays described in detail below), or to generate antibodies specific for PSGL-1 proteins.

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to PSGL-1 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a PSGL-1 nucleic acid molecule of the invention is introduced, e.g., a PSGL-1 nucleic acid molecule within a recombinant expression vector or a PSGL-1 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a PSGL-1 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a PSGL-1 protein. Accordingly, the invention further provides methods for producing a PSGL-1 protein using the host cells of the invention. In one embodiment, the method comprises culturing the host cell of the invention (into which a recombinant expression vector encoding a PSGL-1 protein has been introduced) in a suitable medium such that a PSGL-1 protein is produced. In another embodiment, the method further comprises isolating a PSGL-1 protein from the medium or the host cell.

IV. Methods of Treatment or Prevention of Restenosis and Stenosis:

The present invention provides for both prophylactic and therapeutic methods of treating a subject, e.g., a human, at risk of (or susceptible to) stenosis or restenosis, including constrictive vascular remodeling and neointimal formation, as a result of vascular injury, e.g. injury from PTCA (restenosis) or pathologic injury, e.g., cardiovascular disease (stenosis). With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either the P-selectin antagonists of the present invention or P-selectin ligand modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic and Therapeutic Methods

In one aspect, the invention provides a method for modulating, e.g., inhibiting, stenosis or restenosis in a subject by administering to the subject an agent which modulates PSGL-1 expression or PSGL-1 activity, e.g., modulates P-selectin binding, modulates intercellular adhesion, e.g., platelet-leukocyte adhesion and endothelial-leukocyte adhesion, or modulates cell migration, e.g., leukocyte recruitment to platelets and endothelial cells, modulates restenosis, modulates vascular remodeling, and modulates neointimal formation. Subjects at risk for stenosis or restenosis can be identified by, for example, any or a combination of the diagnostic or prognostic assays described herein. In particular, subjects at risk for stenosis are those individuals who suffer from cardiovascular disease. Subjects who are at risk for restenosis include those who are undergoing cardiovascular and general vascular procedures or intervention such as surgical revascularization, stenting, PCTA or other intervention, surgical or non-surgical, which causes vascular injury.

Cardiovascular diseases and disorders which place a subject at risk for stenosis and make them a target for treatment with the P-selectin antagonists of the invention include arteriosclerosis, ischemia reperfusion injury, arterial inflammation, rapid ventricular pacing, coronary microembolism, tachycardia, bradycardia, pressure overload, aortic bending, vascular heart disease, atrial fibrilation, Jervell syndrome, Lange-Nielsen syndrome, long-QT syndrome, congestive heart failure, sinus node dysfunction, angina, heart failure, hypertension, atrial fibrillation, atrial flutter, cardiomyopathy, e.g., dilated cardiomyopathy and idiopathic cardiomyopathy, myocardial infarction, coronary artery disease, coronary artery spasm, and arrhythmia.

Administration of a prophylactic or theraputic agent, e.g., an anti-P-selectin antibody, an anti-P-selectin ligand antibody, or soluble P-selectin ligand, can occur prior to the manifestation of restenosis or stenosis, or prior to the introduction of vascular injury, such that restenosis or stenosis is inhibited or, alternatively, delayed in its progression, and positive vascular remodeling post-intervention is effectuated. In addition, the agent may be administered to a subject with prior vascular injury caused by vascular or cardiovascular disease who is undergoing vascular intervention resulting in further vascular injury.

Methods of administering to a subject a P-selectin antagonist, e.g., an anti-P-selectin antibody, an anti-P-selectin ligand antibody, soluble P-selectin ligand, soluble PSGL-1 or soluble rPSGL-Ig, to prevent or treat restenosis and positively impact vascular remodeling, include, but are not limited to, the following methods. The soluble P-selectin antagonists of the invention can be administered to a subject using pharmaceutical compositions suitable for such administration. Such compositions typically comprise the agent (e.g., protein or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates PSGL-1 activity (e.g., a fragment of a soluble PSGL-1 protein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate PSGL-1 activity can also be prepared in the form of suppositories (e.g., with conventional suppository bases such as cocoa butter and other glycerides) or retention enemas for rectal delivery.

In one embodiment, the agents that modulate PSGL-1 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates PSGL-1 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such PSGL-1 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (ie., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a therapeutic agent or a radioactive metal ion. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

B. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer a P-selectin antagonist, e.g., soluble PSGL-1, as well as tailoring the dosage and/or therapeutic regimen of treatment with an agent which modulates PSGL-1 activity.

Pharmacogenomics deals with clinically significant hereditary variations in the response to drugs due to altered drug disposition and abnormal action in affected persons. See, for example, Eichelbaum, M. et al. (1996) Clin. Exp.Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a PSGL-1 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a PSGL-1 molecule or P-selectin antagonist of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating or preventing restenosis or stenosis with an agent which modulates PSGL-1 activity.

V. Screening Assays:

The invention provides methods (also referred to herein as “screening assays”) for identifying modulators, i.e., candidate or test compounds or agents (e.g., peptides, peptidomimetics, small molecules, ribozymes, or PSGL-1 antisense molecules) which bind to PSGL-1 proteins, have a stimulatory or inhibitory effect on PSGL-1 expression or PSGL-1 activity, or have a stimulatory or inhibitory effect on the expression or activity of a PSGL-1 target molecule, e.g. P-selectin, or have an effect, e.g., inhibition of cellular migration or adhesion, on cells expressing a PSGL-1 target molecule, e.g., endothelial cells and platelets. Compounds identified using the assays described herein may be useful for modulating stenosis and restenosis, constrictive vascular remodeling, neointimal formation, and cell adhesion and migration.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

Assays that may be used to identify compounds that modulate PSGL-1 activity and P-selectin activity include assays for cell adhesion using ⁵¹Cr-labelled cells, e.g., leukocytes (as described in, for example, Kennedy et al (2000) Br J Pharmacology 130(1):95), and assays for cell migration, e.g., platelet, neutrophil and leukocyte migration (as described in, for example Kogaki et al. (1999) Cardiovascular Res 43(4):968) and Bengtsson et al. (1999) Scand J Clin Lab Invest 59(6):439).

In one aspect, an assay is a cell-based assay in which a cell which expresses a PSGL-1 protein or biologically active portion of the PSGL-1 protein that is believed to be involved in the binding of P-selectin (e.g., amino acid residues 42 to 60 of SEQ ID NO:2) is contacted with a test compound, and the ability of the test compound to modulate PSGL-1 activity is determined. In a preferred embodiment, the biologically active portion of the PSGL-1 protein includes a domain or motif that is capable of interacting with P-selectin or inhibiting P-selectin mediated intercellular adhesion. Determining the ability of the test compound to modulate PSGL-1 activity can be accomplished by monitoring, for example, cell adhesion or cell migration. The cell, for example, can be of mammalian origin, e.g., an endothelial cell, or a leukocyte.

The ability of the test compound to modulate PSGL-1 binding to a substrate or to bind to PSGL-1 can also be determined. Determining the ability of the test compound to modulate PSGL-1 binding to a substrate can be accomplished, for example, by coupling the PSGL-1 substrate with a radioisotope or enzymatic label such that binding of the PSGL-1 substrate to PSGL-1 can be determined by detecting the labeled PSGL-1 substrate in a complex. Alternatively, PSGL-1 could be coupled with a radioisotope or enzymatic label to monitor the ability of a test compound to modulate PSGL-1 binding to a PSGL-1 substrate in a complex. Determining the ability of the test compound to bind PSGL-1 can be accomplished, for example, by coupling the compound with a radioisotope or enzymatic label such that binding of the compound to PSGL-1 can be determined by detecting the labeled PSGL-1 compound in a complex. For example, PSGL-1 substrates can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with PSGL-1 without the labeling of any of the interactants. For example, a microphysiometer can be used to detect the interaction of a compound with PSGL-1 without the labeling of either the compound or the PSGL-1 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and PSGL-1.

In yet another embodiment, an assay of the present invention is a cell-free assay in which a PSGL-1 protein or biologically active portion thereof (e.g., a fragment of a PSGL-1 protein which is capable of binding P-selectin) is contacted with a test compound and the ability of the test compound to bind to or to modulate (e.g., stimulate or inhibit) the activity of the PSGL-1 protein or biologically active portion thereof is determined. Preferred biologically active portions of the PSGL-1 proteins to be used in assays of the present invention include fragments which participate in interactions with non-PSGL-1 molecules, e.g., fragments with high surface probability scores. Binding of the test compound to the PSGL-1 protein can be determined either directly or indirectly as described above. Determining the ability of the PSGL-1 protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In more than one embodiment of the above assay methods of the present invention, it may be desirable to immobilize either PSGL-1 or P-selectin to facilitate separation of complexed from uncomplexed forms of one or both of the proteins, as well as to accommodate automation of the assay. Binding of a test compound to a PSGL-1 protein, or interaction of a PSGL-1 protein with P-selectin in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/PSGL-1 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or PSGL-1 protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of PSGL-1 binding or activity determined using standard techniques.

Other techniques for immobilizing proteins on matrices can also be used in the screening assays of the invention. For example, either a PSGL-1 protein or a P-selectin molecule can be immobilized utilizing conjugation of biotin and streptavidin. Biotinylated PSGL-1 protein or P-selectin protein can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with PSGL-1 protein or P-selectin but which do not interfere with binding of the PSGL-1 protein to its target molecule can be derivatized to the wells of the plate, and unbound target or PSGL-1 protein is trapped in the wells by antibody conjugation. Methods for detecting such complexes, in addition to those described above for the GST-immobilized complexes, include immunodetection of complexes using antibodies reactive with the PSGL-1 protein or P-selectin, as well as enzyme-linked assays which rely on detecting an enzymatic activity associated with the PSGL-1 protein or P-selectin.

In yet another aspect of the invention, the PSGL-1 protein or fragments thereof (e.g., a fragment capable of binding P-selectin) can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with PSGL-1 (“PSGL-1-binding proteins” or “PSGL-1-bp) and are involved in PSGL-1 activity. Such PSGL-1-binding proteins are also likely to be involved in the propagation of signals by the PSGL-1 proteins or PSGL-1 targets as, for example, downstream elements of a PSGL-1-mediated signaling pathway. Alternatively, such PSGL-1-binding proteins are likely to be PSGL-1 inhibitors.

The two-hybrid system is based on the modular nature of most transcription factors, which consist of separable DNA-binding and activation domains. Briefly, the assay utilizes two different DNA constructs. In one construct, the gene that codes for a PSGL-1 protein is fused to a gene encoding the DNA binding domain of a known transcription factor (e.g., GAL4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PSGL-1-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the PSGL-1 protein.

In another aspect, the invention pertains to a combination of two or more of the assays described herein. For example, a modulating agent can be identified using a cell-based or a cell-free assay, and the ability of the agent to modulate the activity of a P-selectin ligand antagonist can be confirmed in vivo, e.g., in an animal such as an animal model for cardiovascular disease. Other examples of animals that may be used include non-recombinant, non-genetic animal models of restenosis or constrictive vascular remodeling such as, for example, rabbit, mouse, porcine, or rat models in which the animal has been subjected to vascular injury, e.g., balloon angioplasty, (as described in Razavi et al (1999) Int J Radiat Oncol Biol Phys 44(2):363-7), injection of a photoactive dye, e.g., rose bengal, (as described in Trieu et al(2000) J Cardiovasc Pharmacol 35(4):595-605), coronary stents (as described in Baumbach et al (2000) Basic Res Cardiol 95(3):173-8), or vascular ligation (as described in Kumar et al (1997) Artheroscler Thromb Vasc Biol. 17:2238). The extent of modulation of restenosis and vascular remodeling can be measured, for example, by morphological analysis of the cardiovascular vessels prior to vascular injury and post-vascular injury. PSGL-1 and P-selectin modulators can be identified where there has been positive vascular remodeling (e.g., lack of constrictive remodeling) and modulation of restenosis (e.g. prevention, inhibition or treatment).

Moreover, a PSGL-1 modulator identified as described herein (e.g., an antisense PSGL-1 nucleic acid molecule, a PSGL-1-specific antibody, or a small molecule) can be used in an animal model to determine the efficacy, toxicity, or side effects of treatment with such a modulator. Alternatively, a PSGL-1 modulator identified as described herein can be used in an animal model to determine the mechanism of action of such a modulator.

This invention is further illustrated by the following examples which should not be construed as limiting. The contents of all references, patents and published patent applications cited throughout this application, as well as the Figures and the Sequence Listing is incorporated herein by reference.

### EXAMPLES

### Example 1

### Construction of Soluble P-Selection Ligands

The EcoRI adaptors used to generate the cDNA library from HL60 cells in Example I contain an XbaI restriction site (TCTAGA) (SEQ ID NO:3) just 5′ of the beginning of SEQ ID NO:1 as it is located in the pMT21:PL85 plasmid. In order to generate soluble forms of the PSL, the pMT21:PL85 plasmid was restricted with XbaI and with HincII (which cleaves after nucleotide 944 of SEQ ID NO:1). The approximately 950 bp fragment thus generated, containing all of the encoded extracellular segment of the ligand up to and including the codon for valine 295, was isolated and used to generate DNAs encoding soluble forms of the P-selectin ligand protein as set forth in sections A though D below.

A. Construction of psPSL.QC

The fragment was purified and ligated into mammalian expression vector pED between the XbaI and EcoRI sites, along with double stranded synthetic oligonucleotide DNA that recreated the codons from Asn 296 to Cys 310 and introduced a novel stop codon immediately following Cys 310. The sequence of the oligos is as follows:

| | |
|---|---|
| 5′-AACTACCCAGTGGGAGCACCAGACCACATCTCTGTGAAGCAGTGCTAG | (SEQ ID NO:4) |
| 5′-AATTCTAGCACTGCTTCACAGAGATGTGGTCTGGTGCTCCCACTGGGTAGTT | (SEQ ID NO:5) |

The resulting plasmid was designated pED.sPSL.QC, and the protein expressed from the plasmid was designated sPSL.QC.

B. Construction of psPSL.Q

The fragment was purified and ligated into the pED plasmid (Kaufman et al., 1991) between the XbaI and EcoRI sites, along with the double stranded synthetic oligonucleotide DNA that recreated the codons from Asn 296 to Gln 309 and introduced a novel stop codon immediately following Gln 309. The sequence of the oligos is as follows:

| | |
|---|---|
| 5′-AACTACCCAGTGGGAGCACCAGACCACATCTCTGTGAAGCAGTAG | (SEQ ID NO:6) |
| 5′-AATTCTACTGCTTCACAGAGATGTGGTCTGGTGCTCCCACTGGGTAGTT | (SEQ ID NO:7) |

The resulting plasmid was designated pED.sPSL.Q, and the protein expressed from the plasmid was designated sPSL.Q.

C. Construction of psPSL.T7

Oligonucleotides encoding 14 amino acids including an epitope derived from the phage T7 major capsid protein were synthesized, creating a C-terminal fusion of the epitope “tag” with an additional 32 amino acids derived from the vector sequence. Two oligonucleotides having the sequences:

| | |
|---|---|
| 5′-CTAGACCCGGGATGGCATCCATGACAGGAGGACAACAAATGGTAGGCCGTAG; and | (SEQ ID NO:8) |
| 5′-AATTCTACGGCCTACCCATTTGTTGTCCTCCTGTCATGGATGCCATCCCGGGT | (SEQ ID NO:9) |

were duplexed and ligated with the large XbaI-EcoRI fragment of mammalian expression plasmid pED. The resulting plasmid, pED.T7 was restricted with XbaI and SmaI and ligated to the 950 bp XbaI-HincII fragment described above, resulting in plasmid pED.sPSL.T7.

The protein resulting from expression of pED.sPSL.T7 was designated sPSL.T7.

D. Construction of Soluble P-selectin Ligand—IgGFc Chimera

The plasmid DNA encoding a soluble, extracellular form of the P-selectin ligand protein fused to the Fc portion of human immunoglobulin IgG1 was constructed as follows: the mammalian expression vector pED.Fc contains sequences encoding the Fc region of a human IgG1 with a novel linker sequence enabling the fusion of coding sequences amino terminal to the hinge region via a unique XbaI restriction site. A three fragment ligation was performed: pED.Fc was restricted with XbaI and gel purified in linear form. The 950 bp fragment from pMT21:PL85 described above comprised the second fragment. The third fragment consisted of annealed synthetic oligonucleotide DNAs having the following sequence:

| | |
|---|---|
| 5′-CTGCGGCCGCAGT | (SEQ ID NO:10) |
| 5′-CTAGACTGCGGCCGCAG | (SEQ ID NO:11) |

The ligation products were grown as plasmid DNAs and individual clones having the correct configuration were identified by DNA sequencing. The plasmid was designated pED.PSL.Fc. The DNA coding region of the resulting soluble P-selectin ligand/Fc fusion protein is shown in SEQ ID NO:12.

### Example 2

### Effect of Soluble P-Selectin Glycoprotein Ligand-1 Chimera on Restenosis Following Arterial Injury by Repeat Angioplasty in Pigs

This example describes the effect of a soluble P-selectin glycoprotein ligand-1 (PSGL-1) chimera (rPSGL-Ig) on restenosis following arterial injury by repeat carotid angioplasty. The repeat carotid injury model is a clinically relevant double injury model in which the first angioplasty creates damage to the vessel similar to the vascular injury caused by vascular intervention or from a cardiovascular disease or disorder. rPSGL-Ig is a recombinant soluble form of PSGL-1 fused to a human IgG (see Example 1D).

Crossbred Yorkshire swine (15-20 kg in weight) were used in the following experiment. Angioplasty of the common carotid arteries using a 7F balloon dilated at 6 atm was performed on both carotid arteries of the pigs used in the experiment. Angiographic measurements of carotid arteries diameter and of balloon/artery ratio were taken. The animals were then allowed to recover for a period of four weeks, during which time neoinitmal lesions developed at injury sites. Repeat angioplasty at the same sites was then performed 15 minutes after a single administration of either a vehicle (formulation buffer), or rPSGL-1-Ig (1 mg/kg, IV, with a half life of 10 days in pigs). Twenty-three animals received the vehicle and 17 animals received rPSGL-Ig just prior to the second angioplasty. Following the second angioplasty, autologous ⁵¹Cr-platelets and ¹¹¹In-neutrophils were radioactively labeled and injected. The animals were sacrificed at 1 hour, 4 hours, 1 week, or 4 weeks following the second angioplasty. Each animal was euthanized under anesthesia with in situ perfusion of carotid arteries.

After the animals were euthanized, a macroscopic examination of dilated and non-dilated carotid arterial segments was performed and ⁵¹Cr-platelet and ¹¹¹In-neutrophil adhesion to arterial segments were quantified using a gama counter. Immunohistochemical detection of P-selection and morphometric analysis of histological arterial sections were also performed to assess restenosis.

Blood sampling, angiograms, ⁵¹Cr-platelet and ¹¹¹In-neutrophil injections were carried out at 1 hr, 4 hrs, 1 week, and 4 weeks after the second angioplasty. Blood sampling and angiograms were also carried out after the first angioplasty of carotid arteries, prior to the second angioplasty, and at the second angioplasty. An evaluation of hematological parameters, hemodynamic parameters, activated clotting time (ACT), and platelet aggregation in whole blood after ADP (10 uM) stimulation, was performed in the animals before treatment with the vehicle or rPSGL-Ig and after treatment with the vehicle or rPSGL-1. The hematological and hemodynamic parameters in control and rPSGL-Ig treated animals are illustrated in Table 1, below.

TABLE 1

| | Vehicle | | rPSGL-Ig | |
|---|---|---|---|---|
| Parameters | Before | After | Before | After |
| Number of Animals | 23 | 23 | 17 | 17 |
| Leukocytes | 19.9 ± 1.3 | 19.2 ± 1.3 | 21.7 ± 2.0 | 20.7 ± 2.1 |
| (× 10⁶/mL) | | | | |
| Neutrophils (%) | 53.3 ± 4.0 | 53.5 ± 3.9 | 53.6 ± 3.7 | 58.4 ± 3.4 |
| Platelets (× 10⁶/mL) | 427 ± 22 | 423 ± 23 | 466 ± 31 | 466 ± 24 |
| Hematrocrit (%) | 26.6 ± 0.6 | 25.8 ± 0.6 | 26.4 ± 0.7 | 25.9 ± 0.7 |
| Activated clotting | 117 ± 3 | 126 ± 5 | 115 ± 2 | 129 ± 6* |
| time (sec) | | | | |
| Heart rate (bpm) | 124 ± 8 | 129 ± 7 | 103 ± 8 | 114 ± 8* |
| Mean arterial | 66 ± 3 | 65 ± 3 | 58 ± 2 | 61 ± 2 |
| pressure (mm Hg) | | | | |

*p < 0.05 vs before, paired student's t-test

Results indicate that rPGSL-Ig reduced adhesion of platelets by 85% (FIG. 1) and neutrophils by 75% (FIG. 2) in deeply injured arterial segments 1 week following repeat angioplasty.

As shown in Table 2, below, at 4 weeks, the residual lumen in deeply injured segments was 63% larger in the rPSGL-Ig treated pigs as compared to the control (6.1±0.6 vs. 3.8±0.1 mm²). The neointimal area in the rPGSL-Ig treated animals was slightly smaller than in the control (0.5±0.1 vs 0.7±0.1 mm²). The ratio of the external elastic lamina (EEL) surface in deeply injured to uninjured vessel segments was 1.5±0.1 in the rPGSL-Ig group vs. 0.9±0.05 in the control group (p<0.01) which indicates a positive effect on compensatory remodeling (see FIGS. 5 and 6).

TABLE 2

| Parameters | Vehicle | rPSGL-Ig |
|---|---|---|
| EEL surface (mm²) | 6.93 ± 0.60 | 10.55 ± 0.85* |
| Normalized | 0.96 + 0.05 | 1.51 + 0.10* |
| EEL Length (mm) | 9.38 ± 0.40 | 11.57 ± 0.44* |
| Normalized | 0.98 + 0.03 | 1.18 + 0.03* |
| IEL surface (mm²) | 4.54 ± 0.60 | 6.60 ± 0.60* |
| Normalized | 0.86 ± 0.08 | 1.34 ± 0.09* |
| IEL length (mm²) | 7.49 ± 0.05 | 9.18 ± 0.04* |
| Normalized | 0.91 ± 0.04 | 1.10 ± 0.03* |
| Residual lumen (mm²) | 3.84 ± 0.06 | 6.08 ± 0.58* |
| Normalized | 0.71 ± 0.09 | 1.22 ± 0.08* |
| % vascular stenosis | 29.22 ± 9.52 | −21.59 ± 8.49* |
| Neointimal surface | 0.70 ± 0.09 | 0.52 ± 0.09 |
| (mm²) | | |

EEL: External elastic lamina
IEL: Internal elastic lamina
Normalized: dilated/reference values
*p < 0.005 vs control

FIG. 6 illustrates the remodeling effect of treatment with rPSGL-Ig 4 weeks after the second angioplasty. Treatment with rPSGL-Ig resulted in a larger lumen area and less neointimal formation, as compared to the control artery. Accordingly, treatment with rPSGL-Ig has resulted in the inhibition of restenosis, as compared to the control.

### Example 3

### Inhibition of In-Stent Restenosis and Neointimal Formation by Soluble P-Selectin Glycoprotein Ligang-1 Chimera in Pigs

This study describes the inhibition of neointimal formation and restenosis following stenting of coronary arteries after angioplasty.

Coronary angioplasty was performed resulting in injury to the LAD, LCX, and RCA coronary arteries of pigs. Two weeks following initial injury, stents were implanted at the injury-induced lesion site in 2 randomly selected vessels. Six pigs (control animals) received a vehicle (formulation buffer) and five pigs received rPSGL-1 (1 mg/kg). The vehicle and rPSGL-1 were administered as a single IV bolus, 15 minutes before stenting. Four weeks later, adhesion of ⁵¹Cr-platelets and ¹¹¹In-neutrophils was quantified and histo-morphologic analysis was performed.

In reference, non-injured artery segments, the vascular lumen was similar in both control (3.0 mm²) and rPSGL-Ig (2.8 mm²) groups. The overall cross sectional area of stented and reference sites was unchanged between groups. However, in-stent residual lumen was reduced significantly by 49% to 1.6±0.4 mm² in control, whereas it remained statistically unchanged (3.2±0.5 mm2) in rPSGL-Ig treated animals, indicating significant inhibition of restenosis by rPSGL-Ig.

Neointimal area as a percentage of total cross sectional area was reduced by rPSGL-Ig treatment (66.7±2.8 vs 52.4±4.9; p<0.05). There was a 49% inhibition of neutrophil adhesion (p<0.05), and a 39% reduction of platelet adhesion.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for modulating stenosis or restenosis in a subject having vascular injury or cardiovascular disease, comprising administering a P-selectin antagonist, thereby modulating stenosis or restenosis.

2. The method of claim 1, wherein the P-selectin antagonist comprises a P-selectin ligand protein, or a fragment thereof having P-selectin ligand activity.

3. The method of claim 1, wherein the P-selectin antagonist comprises a soluble P-selectin ligand protein, or a fragment thereof having P-selectin ligand activity.

4. The method of claim 3, wherein the P-selectin antagonist is soluble PSGL-1.

5. The method of claim 3, wherein the P-selectin antagonist is soluble rPSGL-Ig.

6. The method of claim 1, wherein the P-selectin antagonist comprises an anti-P-selectin antibody.

7. The method of claim 1, wherein the P-selectin antagonist comprises an anti-P-selectin ligand antibody.

8. The method of claims 1, wherein the composition comprises a pharmaceutically acceptable carrier.

9. The method of claim 1, wherein restenosis are characterized by constrictive vascular remodeling.

10. The method of claim 1, wherein restenosis is characterized by neointimal formation.

11. The method of claim 1, wherein said subject is human.

12. The method of claim 1, wherein said P-selectin antagonist is administered to the subject prior to vascular injury.

13. The method of claim 1, wherein said vascular injury or cardiovascular disease affects a coronary artery.

14. The method of claim 1, wherein said vascular injury or cardiovascular disease affects a peripheral artery.

15. The method of claim 14, wherein said artery is a carotid artery.

16. The method of claim 1, wherein said vascular injury results from angioplasty.

17. The method of claim 16, wherein said angioplasty is percutaneous transluminal coronary angioplasty (PTCA).

18. The method of claim 1, wherein said vascular injury results from implantation of a stent or stents.

19. The method of claim 3, wherein said protein comprises a soluble P-selectin ligand protein comprising the amino acid sequence set forth in SEQ ID NO:2 from amino acid 42 to amino acid 60.

20. The method of claim 3, wherein said protein comprises a soluble P-selectin ligand protein comprising the amino acid sequence set forth in SEQ ID NO:2 from amino acid 42 to amino acid 88.

21. The method of claim 3, wherein said protein comprises a soluble P-selectin ligand protein comprising the amino acid sequence set forth in SEQ ID NO:2 from amino acid 42 to amino acid 118.

22. The method of claim 3, wherein said protein comprises a soluble P-selectin ligand protein comprising the amino acid sequence set forth in SEQ ID NO:2 from amino acid 42 to amino acid 189.

23. The method of claim 3, wherein said protein comprises a soluble P-selectin ligand protein comprising the amino acid sequence set forth in SEQ ID NO:2 from amino acid 42 to amino acid 310.

24. The method of claim 3, wherein said protein comprises a soluble P-selectin ligand protein comprising the amino acid sequence set forth in SEQ ID NO:2 from amino acid 42 to amino acid 316.

25. The method of claim 3, wherein said soluble protein comprises an Fc portion of an immunoglobulin.

26. The method of claim 25, wherein said immunoglobulin is human IgG.

27. The method of claim 3, wherein said soluble protein comprises a soluble P-selectin ligand protein comprising the amino acid sequence from amino acid 42 to amino acid 60 of SEQ ID NO:2 fused at its C-terminus to the Fc portion of an immunoglobulin.

28. The method of claim 3, wherein said soluble protein comprises a soluble P-selectin ligand protein comprising the amino acid sequence from amino acid 42 to amino acid 88 of SEQ ID NO:2 fused at its C-terminus to the Fc portion of an immunoglobulin.

29. The method of claim 27 or 28, wherein said amino acid sequence is fused through a linking sequence.

30. A method for modulating leukocyte recruitment in a subject comprising administering soluble PSGL-1, thereby modulating leukocyte recruitment.

31. A method for modulating leukocyte recruitment in a subject comprising administering an anti-P-selectin ligand antibody, thereby modulating leukocyte recruitment.

32. A method for modulating leukocyte recruitment in a subject comprising administering an anti-P-selectin antibody, thereby modulating leukocyte recruitment.

33. A method for inhibiting cell to cell adhesion in a subject comprising administering soluble PSGL-1, thereby inhibiting cell to cell adhesion.

34. A method for inhibiting cell to cell adhesion in a subject comprising administering an anti-P-selectin ligand antibody, thereby inhibiting cell to cell adhesion

35. A method for inhibiting cell to cell adhesion in a subject comprising administering an anti-P-selectin antibody, thereby inhibiting cell to cell adhesion

36. The method of claim 33, 34, or 35, wherein the adhesive cells are selected from the group consisting of leukocytes, platelets, and endothelial cells.

37. The method of claim 33, 34, or 35, wherein the adhesive cells are leukocytes and platelets.

38. A method for inhibiting cell adhesion to blood vessels in a subject comprising administering soluble PSGL-1, thereby inhibiting cell adhesion to blood vessels.

39. A method for inhibiting cell adhesion to blood vessels in a subject comprising administering an anti-P-selectin ligand antibody, thereby inhibiting cell adhesion to blood vessels.

40. A method for inhibiting cell adhesion to blood vessels in a subject comprising administering an anti-P-selectin antibody, thereby inhibiting cell adhesion to blood vessels.

41. The method of claim 38, 39, or 40, wherein the adhesive cells are selected from the group consisting of leukocytes, platelets and endothelial cells.

42. A method for identifying a compound capable of modulating restenosis comprising assaying the ability of the compound to modulate PSGL-1 protein activity, thereby identifying a compound capable of modulating restenosis.

43. The method of claim 42, wherein the ability of the compound to modulate PSGL-1 polypeptide activity is determined by detecting a decrease in intercellular adhesion.

44. The method of claim 42, wherein said cellular adhesion involves leukocytes, endothelial cells, or platelets.

45. The method of claim 42, wherein the ability of the compound to modulate PSGL-1 polypeptide activity is determined by detecting positive vascular remodeling after vascular injury.

46. The method of claim 42, wherein the ability of the compound to modulate PSGL-1 polypeptide activity is determined by detecting a reduction of neointimal formation.
